(19) United States
(12) Patent Application Publication
(10) Pub. No.: US 2024/0226058 A1
(43) Pub. Date: Jul. 11, 2024

(54) STABILIZED CANNABINOID COMPOSITIONS AND METHODS OF PREPARATION THEREOF

(71) Applicant: Cannacraft, Inc., Santa Rosa, CA (US)

(72) Inventors: Matthew W. Elmes, Rohnert Part, CA (US); James P. Pendergast, Santa Rosa, CA (US)

(73) Assignee: Cannacraft, Inc., Santa Rosa, CA (US)

(21) Appl. No.: 18/617,150

(22) Filed: Mar. 26, 2024

Related U.S. Application Data
(63) Continuation of application No. 17/359,768, filed on Jun. 28, 2021, now abandoned.
(60) Provisional application No. 63/049,310, filed on Jul. 8, 2020.

(51) Int. Cl. A61K 31/352; A23D 7/005; A23D 7/06; A23L 2/52; A23L 33/00; A23L 33/105; A61K 9/00; A61K 31/05; A61K 31/22; A61K 31/4045; A61K 31/465; A61K 31/522; A61K 31/5513; A61K 45/06; A61K 47/18; A61K 47/22

(52) U.S. Cl. CPC A61K 31/352; A23D 7/0053; A23D 7/06; A23L 2/52; A23L 33/105; A23L 33/40; A61K 9/0056; A61K 31/05; A61K 31/22; A61K 31/4045; A61K 31/465; A61K 31/522; A61K 31/5513; A61K 45/06; A61K 47/183; A61K 47/22; A23V 2002/00

(57) ABSTRACT

This disclosure provides cannabinoid compositions with improved shelf-life stability, methods of use thereof, and methods of preparation thereof.

### CROSS-REFERENCE TO RELATED APPLICATIONS

This application claims priority under 35 U.S.C. § 119(e) to U.S. Provisional Patent Application No. 63/049,310, filed Jul. 8, 2020. The foregoing application is incorporated by reference herein in its entirety.

### FIELD OF THE INVENTION

This invention relates to stabilized cannabinoid compositions containing one or more antioxidants and methods of preparation thereof.

### BACKGROUND OF THE INVENTION

Emulsification has become a common means of stabilizing lipophilic cannabinoids in an aqueous solution over relatively long time periods. Cannabinoids entrapped in an emulsion particle are subjected to a high degree of water-exposure at the lipid-water interface, which results in relatively fast rates of loss via chemical oxidation. Δ⁹-tetrahydrocannabinol (THC) will transform into cannabinol (CBN) and one or more other unidentified degradation products.

In-house experimental data shows that emulsified THC is prone to drastically faster rates of oxidation compared to its major phytocannabinoid counterpart cannabidiol (CBD). The aqueous base that is infused also plays a large role in the rate of chemical degradation of THC, with polyphenol-rich solutions, such as beer, hop water, and wine, appearing to exhibit the quickest rates of THC oxidation. Possible biological causes (e.g., brewer's yeast) of cannabinoid loss in these beverages were ruled out through experiments using filter-sterilized hop water.

Accordingly, there exists a need for cannabinoid compositions with improved shelf-life stability and methods of preparation thereof.

### SUMMARY OF THE INVENTION

This disclosure addresses the need mentioned above in a number of aspects. In one aspect, this disclosure provides a composition comprising one or more solubilized cannabinoids and at least one antioxidant, a metal chelator, or a combination thereof.

In some embodiments, the composition is formulated as an emulsion. The emulsion comprises a lipid phase and an aqueous phase. The lipid phase comprises at least a fraction of the one or more cannabinoids, and the aqueous phase comprises a first antioxidant.

In some embodiments, the one or more cannabinoids comprise THC. In some embodiments, the THC is in an amount of between about 1 ppm and about 70,000 ppm.

In some embodiments, the first antioxidant comprises ascorbic acid (vitamin C). In some embodiments, the ascorbic acid is in an amount of between about 0.5 mM and about 1 mM of the aqueous phase.

In some embodiments, the aqueous phase further comprises EDTA. In some embodiments, the EDTA is in an amount of between about 1 ppm and about 150 ppm.

In some embodiments, the lipid phase comprises a second antioxidant. In some embodiments, the second antioxidant comprises tocopherol (vitamin E). In some embodiments, the tocopherol is in an amount of between about 2% and about 5% by weight of the lipid phase.

In some embodiments, the composition comprises: (a) between about 0.5 mM and about 1 mM of the ascorbic acid; (b) between about 1 ppm and about 150 ppm of the EDTA; and (c) between about 2% and about 5% by weight of the tocopherol.

In some embodiments, the emulsion comprises emulsion droplets having a mean diameter of between about 50 nm and about 800 nm.

In some embodiments, the one or more cannabinoids are solubilized through cyclodextrin-mediated encapsulation, THC-glycosylation, or a protein-mediated carrier.

In some embodiments, the composition further comprises an additive, a pharmaceutically acceptable carrier, an adjuvant or a second agent. In some embodiments, the second agent is selected from the group consisting of cannabinoids, terpenes, anti-insomnia, anti-tussive, opioid analgesic, decongestant, non-opioid analgesic/anti-inflammatory drug, anti-migraine drug, anti-emetic, anti-histamine, proton pump inhibitor, H2 antagonist/H2 blocker, tranquilizer, anticonvulsant, hypnotic, muscle relaxant, anti-psychotic, anti-diarrheal, attention deficit and hyperactivity disorder (ADHD) drug, anti-Parkinson disease drug, benzodiazepine, benzodiazepine antagonist, barbiturate, barbiturate antagonist, stimulant, stimulant antagonist, antidepressant, nutraceutical, nicotine, BCS Class II active ingredient, BCS Class IV active ingredient, anti-multiple sclerosis (MS) drug, ethyl pyruvate, melatonin, caffeine, resveratrol, and a combination thereof.

In some embodiments, the composition is an oral dosage composition, a pulmonary or nasal dosage composition, or a topical dosage composition.

In another aspect, this disclosure also provides an edible product comprising the composition as described above. In some embodiments, the edible product is selected from a lozenge, candy, chocolate, brownie, cookie, trail bar, cracker, dissolving strip, pastry, bread, chewing gum, mints, pressed candies (like sweet tarts), dissolvable powder formulations of cannabinoids, or droppers/tinctures.

In another aspect, this disclosure further provides a kit comprising the composition as described. In some embodiments, the kit further comprises a beverage, wherein the composition and the beverage are in separate containers.

In yet another aspect, this disclosure additionally provides a method of treating a subject. The method comprises administering to a subject afflicted with or suffering from nausea, muscular spasms, multiple sclerosis, uterine cramps, bowel cramps, a movement disorder, pain, migraine headache, glaucoma, asthma, inflammation, insomnia, high blood pressure, cancer, anxiety, convulsions, depression or psychosis, an effective amount of the composition as described above. In some embodiments, the method comprises administrating the composition to the subject intratumorally, intravenously, subcutaneously, intraosseously, orally, transdermally, in sustained release, in controlled release, in delayed release, as a suppository, or sublingually. In some embodiments, the method comprises administrating the composition to the subject once, twice, three, four times per day or as needed.

In yet another aspect, this disclosure also provides a method for preparing a composition comprising one or more solubilized cannabinoids and at least one antioxidant, a metal chelator, or a combination thereof. The method comprises: (a) providing a solution comprising cannabinoids; and (b) using the solution to generate an emulsion comprising a lipid phase and an aqueous phase, the lipid phase comprising at least a fraction of the one or more cannabinoids and the aqueous phase comprising a first antioxidant.

In some embodiments, the one or more cannabinoids comprise THC. In some embodiments, the first antioxidant comprises ascorbic acid. In some embodiments, the aqueous phase further comprises EDTA. In some embodiments, the lipid phase comprises a second antioxidant. In some embodiments, the second antioxidant comprises tocopherol.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

A clear understanding of the key features of the invention summarized above may be had by reference to the appended drawings, which illustrate the method and system of the invention, although it will be understood that such drawings depict preferred embodiments of the invention and, therefore, are not to be considered as limiting its scope with regard to other embodiments which the invention is capable of contemplating.

FIG. 1 is a graph showing quantified THC content in infused hop water beverages stored at room temperature.

FIG. 2 is a graph showing quantified CBN (the canonical oxidation product of THC) in the room temperature HiFi samples.

FIG. 3 is a graph showing an experiment ruling out any biological cause of cannabinoid loss in beverages.

FIG. 4 is a graph showing an experiment ruling out pH as a driving factor behind THC loss.

FIG. 5 is a graph showing quantified THC content when alpha-tocopherol (Vitamin E, VE) was incorporated into our emulsion alongside THC.

FIG. 6 is a graph showing cannabinoid stability testing on a cold-brewed coffee product (20× concentrated base and final product) using two different emulsions containing tocopherols (QNE=our in-house Q-naturale+VitE emulsion; NGAO=Nanogen's (a 3^{rd} party emulsion provider) antioxidant formulation).

FIG. 7 is a graph showing quantified THC content when natural antioxidant ascorbic acid (Vitamin C, 176 ppm=1 mM) and/or metal chelator EDTA (25 ppm was incorporated in the aqueous phase.

FIG. 8 is a graph showing quantified THC content when natural antioxidant ascorbic acid (Vitamin C, 176 ppm=1 mM) and/or metal chelator EDTA (25 ppm was incorporated in the aqueous phase. Aerated samples were shaken up over a few hours with air exposure to purposely increase dissolved oxygen (DO) levels.

FIG. 9 is a graph showing quantified THC content in infused hop water products when vitamin C or EDTA was incorporated in the aqueous phase.

FIG. 10 is a graph showing combined data from heat-accelerated testing on three different emulsion formulations.

FIG. 11 is a graph showing quantified THC content in Infused hop water products with different concentrations (0, 0.5 mM, 1 mM, 2 mM, and 10 mM) of vitamin C.

FIG. 12 is a graph showing quantified THC content in infused hop water products with 0.5 mM vitamin C.

### DETAILED DESCRIPTION OF THE INVENTION

This disclosure is based, at least in part, to the unexpected discoveries that using a combination of lipid-phase and aqueous-phase antioxidants significantly reduces chemical degradation of emulsified cannabinoids (e.g., THC). Accordingly, this disclosure provides cannabinoid compositions with improved shelf-life stability, methods of use thereof, and methods of preparation thereof.

### A. STABILIZED ANTIOXIDANT-CONTAINING COMPOSITIONS AND METHODS OF USES

In one aspect, this disclosure provides a composition comprising one or more solubilized cannabinoids and at least one antioxidant, a metal chelator or a combination thereof.

In some embodiments, the composition is formulated as an emulsion. The emulsion comprises a lipid phase and an aqueous phase. The lipid phase comprises at least a fraction of the one or more cannabinoids, and the aqueous phase comprises a first antioxidant.

In some embodiments, the cannabinoids, such as THC, are processed from Cannabis sativa or Cannabis indica. The cannabis plant material may or may not need to be pre-processed. For example, the raw cannabis plant material can be used directly for cannabis extraction. In some embodiments, the method further comprises grinding Cannabis sativa or Cannabis indica into ground cannabis plant material.

As used herein, “Cannabis sativa L.” or “Cannabis sativa” refers to an annual herbaceous plant in the Cannabis genus, a species of the Cannabaceae family. As used herein, “Cannabis indica Lam” or “Cannabis indica” refers to an annual plant in the Cannabaceae family. A putative species of the genus Cannabis, it is typically distinguished from Cannabis sativa. Cannabis sativa and Cannabis indica can interbreed, so the two strains can be viewed as sub-species or landraces. Interbred stains comprising genetic material from both sativa and indica strains can be termed “sativa-dominant” or “indica-dominant,” depending upon perceived physical and psychotropic properties of the hybrids. The mixed interbred strains can be themselves reproductively viable.

As used herein, “Cannabis ruderalis Janisch” or “Cannabis ruderalis” refers to a species of Cannabis originating in central Russia. It flowers earlier than C. indica or C. sativa, does not grow as tall, and can withstand much harsher climates than either of them. Cannabis ruderalis will produce flowers based on its age, rather than light cycle (photoperiod) changes which govern flowering in C. sativa and C. indica varieties. This kind of flowering is also known as “autoflowering.”

As used herein, “Cannabis” refers to a genus of flowering plants that includes a single species, Cannabis sativa, which is sometimes divided into two additional species, Cannabis indica and Cannabis ruderalis. These three taxa are indigenous to Central Asia, and South Asia. Cannabis has long been used for fiber (hemp), for seed and seed oils, for medicinal purposes, and as a recreational drug. Various extracts including hashish and hash oil are also produced from the plant. Suitable strains of Cannabis include, e.g., Indica-dominant (e.g., Blueberry, BC Bud, Holland's Hope, Kush, Northern Lights, Purple, and White Widow), Pure sativa (e.g., Acapulco Gold and Malawi Gold (Chamba)), and Sativa-dominant (e.g., Charlotte's Web, Diesel, Haze, Jack Herer, Shaman, Skunk, Sour, and Te Puke Thunder). Cannabis plant material can include any physical part of the plant material, including, e.g., the leaf, bud, flower, trichome, seed, or combination thereof. Likewise, the cannabis plant material can include any substance physically derived from cannabis plant material, e.g., kief and hashish.

In some embodiments, the one or more cannabinoids comprise THC. In some embodiments, the THC is in an amount of between about 1 ppm and about 70,000 ppm.

In some embodiments, THC can comprise delta-9-THC, delta-8-THC, and combinations thereof. THC can comprise delta-6a,7-tetrahydrocannabinol, delta-7-tetrahydrocannabinol, delta-8-tetrahydrocannabinol, delta-9,11-tetrahydrocannabinol, delta-9-tetrahydrocannabinol, delta-10-tetrahydrocannabinol, delta-6a,10a-tetrahydrocannabinol, and combinations thereof. Delta-9-tetrahydrocannabinol can comprise stereoisomers including (6aR,10aR)-delta-9-tetrahydrocannabinol, (6aS,10aR)-delta-9-tetrahydrocannabinol, (6aS,10aS)-delta-9-tetrahydrocannabinol, (6aR,10aS)-delta-9-tetrahydrocannabinol, THCV, THCP, and combinations thereof.

In some embodiments, the first antioxidant comprises ascorbic acid (vitamin C). In some embodiments, the ascorbic acid is in an amount of between about 0.5 mM and about 1 mM of the aqueous phase.

In some embodiments, the aqueous phase further comprises EDTA. In some embodiments, the EDTA is in an amount of between about 1 ppm and about 150 ppm.

In some embodiments, the lipid phase comprises a second antioxidant. In some embodiments, the second antioxidant comprises tocopherol (vitamin E). In some embodiments, the tocopherol is in an amount of between about 2% and about 5% by weight of the lipid phase. In some embodiments, the composition comprises: (a) between about 0.5 mM and about 1 mM of the ascorbic acid; (b) between about 1 ppm and about 150 ppm; and (c) between about 2% and about 5% by weight of the tocopherol.

In some embodiments, the emulsion comprises emulsion droplets having a mean diameter of between about 50 nm and about 800 nm.

In some embodiments, the one or more cannabinoids are solubilized through cyclodextrin-mediated encapsulation, THC-glycosylation, or a protein-mediated carrier.

In some embodiments, the composition further comprises an additive, a pharmaceutically acceptable carrier, an adjuvant or a second agent. In some embodiments, the second agent is selected from the group consisting of: cannabinoids, terpenes, anti-insomnia, anti-tussive, opioid analgesic, decongestant, non-opioid analgesic/anti-inflammatory drug, anti-migraine drug, anti-emetic, anti-histamine, proton pump inhibitor, H2 antagonist/H2 blocker, tranquilizer, anticonvulsant, hypnotic, muscle relaxant, anti-psychotic, anti-diarrheal, attention deficit and hyperactivity disorder (ADHD) drug, anti-Parkinson disease drug, benzodiazepine, benzodiazepine antagonist, barbiturate, barbiturate antagonist, stimulant, stimulant antagonist, antidepressant, nutraceutical, nicotine, BCS Class II active ingredient, BCS Class IV active ingredient, anti-multiple sclerosis (MS) drug, ethyl pyruvate, melatonin, caffeine, resveratrol, and a combination thereof.

The compositions of the present disclosure can be provided as a food composition in combination with a food carrier, including but not limited to food bars (e.g., granola bars, protein bars, candy bars), cereal products (e.g., oatmeal, breakfast cereals, granola), bakery products (e.g., bread, donuts, crackers, bagels, pastries, cakes), dairy products (e.g., milk, yogurt, cheese), beverages (e.g., milk-based beverages, sports drinks, fruit juices, teas, soft drinks, alcoholic beverages, bottled waters), beverage mixes, pastas, grains (e.g., rice, corn, oats, rye, wheat, flour), egg products, snacks (e.g., candy, chips, gum, gummies, lozenges, mints, chocolate), meats, fruits, vegetables or combinations thereof. Food compositions can comprise solid foods. Food compositions can comprise semi-solid foods. Food compositions can comprise liquid foods. A composition in a liquid form may be formulated from a dry mix, such as a dry beverage mix or a powder. A dry mix may be suitable in terms of transportation, storage, or shelf life. The composition can be formulated from the dry mix in any suitable manner, such as by adding a suitable liquid (e.g., water, milk, fruit juice, tea, or alcohol).

A food composition or food product can comprise a food bar, including but not limited to, granola bars, protein bars, candy bars, and energy bars. A food composition or food product can comprise a cereal product, including but not limited to oatmeal, flour (e.g., wheat flour, rice flour, cornflour, barley flour), breakfast cereal, granola, bread, pasta, rice cakes, and popcorn. A food composition or food product can comprise a bakery product, including but not limited to bread, pastries, brownies, cakes, pies, donuts, crackers, and muffins. A food composition or food product can comprise a dairy product, including but not limited to milk, fermented milk, curd, whey, yogurt, cream, cheese, butter, clarified butter, ghee, and ice cream. A food composition or food product can comprise a nut butter or seed butter, including but not limited to peanut butter, almond butter, cashew butter, hazelnut butter, macadamia nut butter, pecan butter, pistachio butter, walnut butter, pumpkin seed butter, sesame seed butter, soybean butter, and sunflower seed butter. A food composition or food product can comprise an oil (e.g., a cooking oil), including but not limited to olive oil, coconut oil, vegetable oil, canola oil, corn oil, peanut oil, sunflower seed oil, almond oil, avocado oil, rice bran oil, cottonseed oil, flaxseed oil, linseed oil, grape seed oil, hemp oil, mustard oil, macadamia oil, palm oil, tea seed oil, walnut oil, margarine, lard, butter, clarified butter, ghee, or tallow. A food composition or food product can comprise sports food products such as energy gels, sports drinks, energy powders, energy bars, energy shots, protein powders, and protein drinks (e.g., protein shakes). A food composition or food product can comprise a beverage, including but not limited to water, electrolyte drinks, soda, coconut water, tea (e.g., Jun tea, black tea, green tea, white tea, herbal tea), coffee, a soft drink, an alcoholic beverage (e.g., cocktail, liquor, spirits, beer, wine, malt beverage), water, juice (e.g., apple juice, orange juice, tomato juice, vegetable juice, cranberry juice), a sports drink, electrolyte-enriched water, vitamin-enhanced water, a hangover-recovery drink, milk (e.g., dairy-based milk, coconut milk, almond milk, soy milk, hemp milk, rice milk, oat milk, cashew milk, hazelnut milk), and yogurt. A food composition or food product can comprise a fungus or fermented food or drink, including but not limited to kifir (kefir), jun, amasi, amazake, appam, ayran, doogh, bagoong, brem, cheonggukjang, chicha, kombucha, fermented bean curd, kimchi, lassi, miso, poi, yakult, and yogurt.

Compositions of the present disclosure can comprise pet or other animal products, such as animal food (e.g., dog food, cat food), treats, and nutritional supplements (e.g., liquids, sprays, or powders for application to food or water). These compositions can be formulated for or administered to domestic or pet animals (e.g., dogs, cats, small mammals, birds), livestock, and other farm animals (e.g., cows, pigs, horses, sheep, goats), zoo animals, or any other vertebrates. Compositions for administration to animals can be formulated with microencapsulated cannabinoid-rich oil or non-encapsulated cannabinoid-rich oil, alone or in combination with essential oils, terpenes, and other components described herein. Compositions for administration to animals can be mixed into feed or water, prepared for spraying application (e.g., mixed in glycerin), for intravenous administration (e.g., in a syringe or an IV bag), in salves, vitamins, liquid vitamin pumps, treats, or other forms.

In another aspect, this disclosure additionally provides a method of treating a subject. The method comprises administering to a subject afflicted with or suffering from nausea, muscular spasms, multiple sclerosis, uterine cramps, bowel cramps, a movement disorder, pain, migraine headache, glaucoma, asthma, inflammation, insomnia, high blood pressure, cancer, anxiety, convulsions, depression or psychosis, an effective amount of the composition as described above. In some embodiments, the method comprises administrating the composition to the subject intratumorally, intravenously, subcutaneously, intraosseously, orally, transdermally, in sustained release, in controlled release, in delayed release, as a suppository, or sublingually. In some embodiments, the method comprises administrating the composition to the subject once, twice, three, or four times per day, or as needed.

In some embodiments, the composition is an oral dosage composition, a pulmonary or nasal dosage composition, or a topical dosage composition.

In some embodiments, the compositions as described herein are administered via a vaporizer or like device as described, for example, in U.S. Pat. No. 8,915,254; U.S. Pat. Appl. Pub. No. 2014/0060552; U.S. Pat. No. 8,488,952; and U.S. Pat. Appl. Pub. No. 2015/0040926. Compositions for pulmonary administration also include, but are not limited to, dry powder compositions consisting of the powder of a cannabis oil described herein, and the powder of a suitable carrier and/or lubricant. The compositions for pulmonary administration can be inhaled from any suitable dry powder inhaler device known to a person skilled in the art. In certain instances, the compositions may be conveniently delivered in the form of an aerosol spray from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound(s) and a suitable powder base, for example, lactose or starch.

Pharmaceutical compositions or medicaments can be formulated by standard techniques or methods well-known in the art of pharmacy using one or more physiologically acceptable carriers or excipients. Suitable pharmaceutical carriers are described herein and in, e.g., “Remington's Pharmaceutical Sciences” by E. W. Martin. Cannabis oil extracts can be formulated for administration by any suitable route, including, but not limited to, orally, topically, nasally, rectally, vaginally, pulmonary, parenterally (e.g., intravenously, subcutaneously, intramuscularly, etc.), and combinations thereof. In some embodiments, the cannabis oil is diluted in a liquid, e.g., a carrier oil. The most suitable route of administration in any given case will depend in part on the condition being treated as well as the response of the subject to the particular route of treatment.

For oral administration, a pharmaceutical composition or a medicament can take the form of, e.g., a tablet or a capsule prepared by conventional means with a pharmaceutically acceptable excipient. Preferred are tablets and gelatin capsules comprising the active ingredient(s), together with (a) diluents or fillers, e.g., lactose, dextrose, sucrose, mannitol, maltodextrin, lecithin, agarose, xanthan gum, guar gum, sorbitol, cellulose (e.g., ethyl cellulose, microcrystalline cellulose), glycine, pectin, polyacrylates and/or calcium hydrogen phosphate, calcium sulfate, (b) lubricants; e.g., silica, anhydrous colloidal silica, talcum, stearic acid, its magnesium or calcium salt (e.g., magnesium stearate or calcium stearate), metallic stearates, colloidal silicon dioxide, hydrogenated vegetable oil, corn starch, sodium benzoate, sodium acetate and/or polyethyleneglycol; for tablets also (c) binders, e.g., magnesium aluminum silicate, starch paste, gelatin, tragacanth, methylcellulose, sodium carboxymethylcellulose, polyvinylpyrrolidone and/or hydroxypropyl methylcellulose; if desired (d) disintegrants, e.g., starches (e.g., potato starch or sodium starch), glycolate, agar, alginic acid or its sodium or potassium salt, or effervescent mixtures; (e) wetting agents, e.g., sodium lauryl sulfate, and/or (f) absorbents, colorants, flavors, and sweeteners. Tablets can be either uncoated or coated according to methods known in the art. The excipients described herein can also be used for preparation of buccal dosage forms and sublingual dosage forms (e.g., films and lozenges) as described, for example, in U.S. Pat. Nos. 5,981,552 and 8,475,832. Formulation in chewing gums as described, for example, in U.S. Pat. No. 8,722,022, is also contemplated.

Further preparations for oral administration can take the form of, for example, solutions, syrups, suspensions, and toothpastes. Liquid preparations for oral administration can be prepared by conventional means with pharmaceutically acceptable additives, for example, suspending agents, for example, sorbitol syrup, cellulose derivatives, or hydrogenated edible fats; emulsifying agents, for example, lecithin, xanthan gum, or acacia; non-aqueous vehicles, for example, almond oil, sesame oil, hemp seed oil, fish oil, oily esters, ethyl alcohol, or fractionated vegetable oils; and preservatives, for example, methyl or propyl-p-hydroxybenzoate or sorbic acid. The preparations can also contain buffer salts, flavoring, coloring, and/or sweetening agents as appropriate.

Typical formulations for topical administration include creams, ointments, sprays, lotions, hydrocolloid dressings, and patches, as well as eye drops, ear drops, and deodorants. Cannabis oils can be administered via transdermal patches as described, for example, in U.S. Pat. Appl. Pub. No. 2015/0126595 and U.S. Pat. No. 8,449,908. Formulation for rectal or vaginal administration is also contemplated. The cannabis oils can be formulated, for example, using suppositories containing conventional suppository bases such as cocoa butter and other glycerides as described in U.S. Pat. Nos. 5,508,037 and 4,933,363. Compositions can contain other solidifying agents such as shea butter, beeswax, kokum butter, mango butter, illipe butter, tamanu butter, carnauba wax, emulsifying wax, soy wax, castor wax, rice bran wax, and candelilla wax. Compositions can further include clays (e.g., Bentonite, French green clays, Fuller's earth, Rhassoul clay, white kaolin clay) and salts (e.g., sea salt, Himalayan pink salt, and magnesium salts such as Epsom salt).

The compositions set forth herein can be formulated for parenteral administration by injection, for example, by bolus injection or continuous infusion. Formulations for injection can be presented in unit dosage form, for example, in ampoules or in multi-dose containers, optionally with an added preservative. Injectable compositions are preferably aqueous isotonic solutions or suspensions, and suppositories are preferably prepared from fatty emulsions or suspensions. The compositions may be sterilized and/or contain adjuvants, such as preserving, stabilizing, wetting or emulsifying agents, solution promoters, salts for regulating the osmotic pressure, buffers, and/or other ingredients. Alternatively, the compositions can be in powder form for reconstitution with a suitable vehicle, for example, a carrier oil, before use. In addition, the compositions may also contain other therapeutic agents or substances.

In some embodiments, the composition is an oral dosage composition, a pulmonary or nasal dosage composition, or a topical dosage composition. The composition may be in the form of a solution, a spray, or a powder, a tablet, a capsule, a jelly, a cream, an ointment, a suspension, a spray, or a chewing gum.

In some embodiments, the composition may further comprise a second agent selected from the group consisting of: cannabinoids, terpenes, anti-insomnia, anti-tussive, opioid analgesic, decongestant, non-opioid analgesic/anti-inflammatory drug, anti-migraine drug, anti-emetic, anti-histamine, proton pump inhibitor, H2 antagonist/H2 blocker, tranquilizer, anticonvulsant, hypnotic, muscle relaxant, anti-psychotic, anti-diarrheal, Attention Deficit and Hyperactivity Disorder (ADHD) drug, anti-Parkinson disease drug, benzodiazepine, benzodiazepine antagonist, barbiturate, barbiturate antagonist, stimulant, stimulant antagonist, antidepressant, nutraceutical, nicotine, BCS Class II active ingredient, BCS Class IV active ingredient, an anti-multiple sclerosis (MS) drug, ethyl pyruvate, melatonin, caffeine, resveratrol, and a combination thereof. In some embodiments, the second agent is selected from the group consisting of: CBD, THC, CBN, CBG, CBC, THCA, CBDA, THCV, and a combination thereof.

In some embodiments, the composition at therapeutically effective concentrations or dosages be combined with a pharmaceutically or pharmacologically acceptable carrier, excipient or diluent, either biodegradable or non-biodegradable.

For example, the composition may be administered in the pure form or in a pharmaceutically acceptable formulation including suitable elixirs, binders, and the like (also generally referred to a “carriers”) or as pharmaceutically acceptable salts (e.g., alkali metal salts such as sodium, potassium, calcium or lithium salts, ammonium, etc.) or other complexes. It should be understood that the pharmaceutically acceptable formulations include liquid and solid materials conventionally utilized to prepare both injectable dosage forms and solid dosage forms such as tablets and capsules and aerosolized dosage forms. In addition, the compounds may be formulated with aqueous or oil-based vehicles. Water may be used as the carrier for the preparation of compositions (e.g., injectable compositions), which may also include conventional buffers and agents to render the composition isotonic. Other potential additives and other materials (preferably those which are generally regarded as safe [GRAS]) include: colorants; flavorings; surfactants (TWEEN, oleic acid, etc.); solvents, stabilizers, elixirs, and binders or encapsulants (lactose, liposomes, etc.). Solid diluents and excipients include lactose, starch, conventional disintegrating agents, coatings, and the like. Preservatives such as methylparaben or benzalkonium chloride may also be used. Depending on the formulation, it is expected that the active composition will consist of about 1% to about 99% of the composition and the vehicular “carrier” will constitute about 1% to about 99% of the composition. The pharmaceutical compositions of the present invention may include any suitable pharmaceutically acceptable additives or adjuncts to the extent that they do not hinder or interfere with the therapeutic effect of the active compound.

Examples of carriers include, but are by no means limited to, for example, poly(ethylene-vinyl acetate), copolymers of lactic acid and glycolic acid, poly(lactic acid), gelatin, collagen matrices, polysaccharides, poly(D,L lactide), poly(malic acid), poly(caprolactone), celluloses, albumin, starch, casein, dextran, polyesters, ethanol, methacrylate, polyurethane, polyethylene, vinyl polymers, glycols, mixtures thereof and the like. Standard excipients include gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidal silicon dioxide, phosphates, sodium dodecyl sulfate, carboxymethylcellulose calcium, carboxymethylcellulose sodium, methylcellulose, hydroxyethylcellulose, hydroxypropyl cellulose, hydroxypropyl-methylcellulose phthalate, noncrystalline cellulose, magnesium aluminum silicate, triethanolamine, polyvinyl alcohol, polyvinylpyrrolidone, sugars, and starches. See, for example, Remington: The Science and Practice of Pharmacy, 1995, Gennaro ed.

In some embodiments, the chemicals can be purified and blended together to produce a formulation similar in form to that for Marinol®. In these formulations, the active ingredient is dissolved in sesame seed oil or a similar oil and enclosed in a gel-capsule. In other embodiments, the formulation may be arranged to be used as an injectable or as an aerosol. In these embodiments, as will be apparent to one of skill in the art, the appropriate pharmaceutically-acceptable additives may be added so that the pharmaceutical composition is in the appropriate form.

As will be appreciated by one knowledgeable in the art, the formulation may be used as, for example, an anti-emetic, appetite stimulant, or as a treatment for nausea, dementia, Alzheimer's disease, glaucoma, high blood pressure, inflammation or multiple sclerosis. For example, when administered to an individual in need of such treatment, the pharmaceutical composition of Δ⁸-THC and CBD will accomplish at least one of the following: reduce nausea, promote or stimulate appetite, reduce vomiting and/or promote a general feeling of well-being.

### Additional Ingredients

Cannabinoids are susceptible to oxidation and hydrolysis. Over time it is possible for cannabinoids to be exposed to oxygen, hydrogen ions (acids, water), in addition to any other environmental factors that will cause their degradation.

Organic bases can be used to prevent the degradation of the cannabinoids. These organic bases include, but are not limited to, butyl hydroxyl anisole (BHA), butyl hydroxyl toluene (BHT) and sodium ascorbate; at concentrations between 0.001 to 5%>w/w, for example. Organic bases such as the following can improve the stability of cannabinoids from chemical degradation for up to 2 years: BHA 0.001 to 5% w/w, BHT 0.001 to 5% w/w, and combinations of BHA and BHT can also be used.

Antioxidants can be used to prevent or at least inhibit or mitigate the degradation of cannabinoids from oxidation. Examples of antioxidants include: ethanol, polyethylene glycol 300, polyethylene glycol 400, propylene glycol, propylene carbonate, N-methyl-2-pyrrolidones, dimethylacetamide, dimethyl sulfoxide, hydroxypropyl-P-cyclodextrins, sulfobutylether-β-cyclodextrin, a-cyclodextrin, HSPC phospholipid, DSPG phospholipid, DMPC phospholipid, DMPG phospholipid, ascorbyl palmitate, butylated hydroxyanisole, butylated hydroxyanisole, propyl gallate, a-tocopherol, γ-tocopherol, propyl gallate, lecithin, Vitamin E tocopherol, sesamin, sesamol, sesamolin, alpha-tocopherol, ascorbic acid, ascorbyl palmitate, fumaric acid, malic acid, sodium metabisulfite and EDTA. Specific antioxidant examples include, but are not limited to: Ascorbic Acid: 0.001 to 5% w/w, Vitamin E Tocopherol: 0.001 to 5% w/w, Tocopherol: 0.001 to 5% w/w, and combinations of ascorbic acid, vitamin E tocopherol, and tocopherol can be used for this invention.

Chelating agents can prevent or at least mitigate the degradation of cannabinoids from metal ions in solution. Chelating agents include, but are not limited to, ethylenediaminetetraacetic acid (EDTA), phosphoric acid, polyphosphates, polysaccharides, citric acid and any combination thereof.

Preservatives can be used to prevent microbial spoilage. These preservatives include: methylparabens, ethylparabens, propylparabens, butylparabens, sorbic acid, acetic acid, propionic acid, sulfites, nitrites, sodium sorbate, potassium sorbate, calcium sorbate, benzoic acid, sodium benzoate, potassium benzoate, calcium benzoate, sodium metabisulfite, propylene glycol, benzaldehyde, butylated hydroxytoluene, butylated hydroxyanisole, formaldehyde donors, essential oils, citric acid, monoglyceride, phenol, mercury components and any combination thereof. Specific examples include, but are not limited to, sodium benzoate and potassium sorbate.

Additionally, the pH can be lowered to prevent or retard microbial growth. Lowering the pH below 4.0 is sufficiently low enough to prevent microbial growth for a minimum of 1 month. In some embodiments, the pH of the composition is about 3.6.

Preservatives and/or stabilizers can be added during formulation. Depending on the nature of the preservative/stabilizer, it may be contained in either the oil phase, interfacial layer, or the aqueous continuous phase. Once dissolved the preservatives and stabilizers are released into solution imparting their properties into the aqueous system. This allows beverage manufacturers the ability to instantly create shelf-stable cannabis-infused beverages. Beverages made this way can resist microbial growth and chemical degradation for a minimum of 3 months.

The composition can be used for treatment of a subject afflicted with or suffering from nausea, muscular spasms, multiple sclerosis, uterine cramps, bowel cramps, a movement disorder, pain, migraine headache, vertigo, glaucoma, asthma, inflammation, insomnia, high blood pressure, cancer, anxiety, convulsions, depression or psychosis.

In one aspect, this disclosure provides a kit comprising the composition as described above. In some embodiments, the kit further comprising a beverage, wherein the composition and the beverage are in separate containers. In some embodiments, the kit may further include instructional materials.

“Instructional material,” as used herein, includes a publication, a recording, a diagram, or any other medium of expression that can be used to communicate the usefulness of any composition and/or compound of the invention in a kit. The instructional material of the kit may, for example, be affixed to a container that contains any composition of the invention or be shipped together with a container which contains any composition. Alternatively, the instructional material may be shipped separately from the container with the intention that the recipient uses the instructional material and any composition cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

### B. METHODS OF USES

Accordingly, in another aspect, this disclosure provides a method of treatment of a subject. The method comprises administering to a subject afflicted with or suffering from nausea, muscular spasms, multiple sclerosis, uterine cramps, bowel cramps, a movement disorder, pain, migraine headache, vertigo, glaucoma, asthma, inflammation, insomnia, high blood pressure, cancer, anxiety, convulsions, depression or psychosis, an effective amount of the composition as described above.

Subjects of the present disclosure can include humans and other animals, such as pets (e.g., dogs, cats, birds, small mammals, snakes) and livestock or farm animals (e.g., cows, pigs, horses, sheep, chickens). Compositions of the present disclosure can be useful for veterinary applications.

The compositions of the present disclosure can be administered to a subject. Compositions can be administered in a variety of ways, including but not limited to oral and topical administration.

Administering the compositions of the present disclosure to a subject can provide one or more beneficial effects. Beneficial effects can include but are not limited to pain relief, reduced bacterial growth, reduced blood sugar levels, improved blood lipid and cholesterol profiles, increased fat burning, reduced appetite, stimulated appetite, reduced vomiting or nausea, reduced seizures or convulsions, antifungal effects, reduced inflammation, reduced arthritis (e.g., rheumatoid arthritis), reduced insomnia or aided sleep, reduced arterial blockage, inhibited cancer cell growth, improved psoriasis, tranquilizing effects, antispasmodic effects, reduced anxiety, bone growth promotion, reduced intestinal contractions, and nervous system protection.

Any of the compositions can be provided in a unit dosage form. A unit dosage is an amount of a compound, such as a cannabinoid compound delivered alone or in combination with other components, which is to be administered to a subject at or about one time point. Other components which can be included with a unit dosage include but are not limited to cosmetics, food carriers, food bars, baked goods, dairy products, oils, beverages, solid dosages (e.g., tablets), or liquid dosages. A unit dosage of a cannabinoid compound can be about 10, 20, 30, 40, 50, 60, 70, 80, 90, 100, 125, 150, 175, 200, 250, 300, 350, 400, 450, 500, 600, 700, 800, 900, 1000 or more milligrams (mg). A unit dosage of a cannabinoid compound can be at least about 10, 20, 30, 40, 50, 60, 70, 80, 90, 100, 125, 150, 175, 200, 250, 300, 350, 400, 450, 500, 600, 700, 800, 900, 1000 or more milligrams (mg). A unit dosage of a cannabinoid compound can be at most about 10, 20, 30, 40, 50, 60, 70, 80, 90, 100, 125, 150, 175, 200, 250, 300, 350, 400, 450, 500, 600, 700, 800, 900, 1000 or more milligrams (mg). A unit dosage can be an hourly dosage. A unit dosage can be a daily dosage. A unit dosage can provide about 1/24, 1/12, ⅛, ⅙, ¼, ⅓, ½, or all of a daily dosage of one or more cannabinoids for a subject. A unit dosage can take the form of a tablet, gel, liquid, food product, food bar, container of liquid of defined volume, or other forms described herein, packaged for one-time consumption or administration.

In some embodiments, the composition is administered intratumorally, intravenously, subcutaneously, intraosseously, orally, transdermally, in sustained release, in controlled release, in delayed release, as a suppository, or sublingually. In some embodiments, the composition is administered once, twice, three, or four times per day, or as needed.

### C. METHODS OF PREPARATION

In yet another aspect, this disclosure also provides a method for preparing a composition comprising one or more solubilized cannabinoids and at least one antioxidant. The method comprises: (a) providing a solution comprising cannabinoids; and (b) using the solution to generate an emulsion comprising a lipid phase and an aqueous phase, the lipid phase comprising at least a fraction of the one or more cannabinoids and the aqueous phase comprising a first antioxidant.

In some embodiments, the one or more cannabinoids comprise THC. In some embodiments, the first antioxidant comprises ascorbic acid. In some embodiments, the aqueous phase further comprises EDTA. In some embodiments, the lipid phase comprises a second antioxidant. In some embodiments, the second antioxidant comprises tocopherol.

### D. Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

The term “cannabis” refers to plants of the genus Cannabis, including Cannabis saliva, Cannabis indica, and Cannabis ruderalis.

The term “cannabis oil” refers to a mixture of compounds obtained from the extraction of cannabis plants. Such compounds include, but are not limited to, cannabinoids, terpenes, terpenoids, and other compounds found in the cannabis plant. The exact composition of cannabis oil will depend on the strain of cannabis that is used for extraction, the efficiency and process of the extraction itself, and any additives that might be incorporated to alter the palatability or improve administration of the cannabis oil.

The term “cannabinoid” includes but are not limited to cannabigerol-type (CBG), cannabigerolic acid (CBGA), cannabigerolic acid monomethylether (CBGAM), cannabigerol monomethyl ether (CBGM), cannabichromene-type (CBC), cannabichromanon (CBCN), cannabichromenic acid (CBCA), cannabichromevarin-type (CBCV), cannabichromevarinic acid (CBCVA), cannabidiol-type (CBD), tetrahydrocannabinol-type (THC), iso-tetrahydrocannabinol-type (iso-THC), cannabinol-type (CBN), cannabinolic acid (CBNA), cannabinol methylether (CBNM), cannabinol-C₄ (CBN-C₄), cannabinol-C₂ (CBN-C₂), cannabiorcol (CBN-C₁), cannabinodiol (CBND), cannabielsoin-type (CBE), cannabielsoic acid A (CBEA-A), cannabielsoic acid B (CBEA-B), cannabicyclol-type (CBL), cannabicyclolic acid (CBLA), cannabicyclovarin (CBLV), cannabicitran-type (CBT), cannabitriol, cannabitriolvarin (CBTV), ethoxy-cannabitiolvarin (CBTVE), cannabivarin-type (CBV), cannabinodivarin (CBVD), tetrahydrocannabivarin-type (THCV), cannabidivarin-type (CBDV), cannabigerovarin-type (CBGV), cannabigerovarinic acid (CBGVA), cannabifuran (CBF), dehydrocannabifuran (DCBF), cannabiripsol (CBR) cannabinoids, tetrahydrocannabiphorol (THCP), and cannabidiphorol (CBDP).

As used herein, CBD refers to cannabidiol.

As used herein, Δ⁸-THC, Δ⁹-THC, Δ¹⁰-THC refer to Δ⁸-tetrahydrocannabinol, Δ⁹-tetrahydrocannabinol, and Δ¹⁰-tetrahydrocannabinol, respectively.

As used herein, Δ⁸-THC refers to Δ⁸-tetrahydrocannabinol.

The term “acidic cannabinoid” refers to a cannabinoid having one or more carboxylic acid functional groups. Examples of acidic cannabinoids include, but are not limited to, tetrahydrocannabinolic acid (THCA), cannabidiolic acid (CBDA), and cannabichromenic acid (CBCA). Acidic cannabinoids are frequently the predominant cannabinoids found in raw (i.e., unprocessed) cannabis plant material.

The term “essential oil” refers to natural plant oil typically obtained by distillation and having a chemical composition and organoleptic properties (e.g., fragrance) characteristic of the plant or other sources from which it is extracted.

As used herein, “anti-emetic” refers to compounds capable of reducing nausea, enhancing appetite and/or reducing vomiting in an individual.

By “water-soluble” we mean that 1 mg of material in 1 ml of water gives a clear solution and is water-miscible.

By “high affinity” we mean that the compounds exhibit a Ki in the range of about 0.03 nM to about 80 nM, and preferably from about 0.03 nM to about 50 nM, for either the CB1 or CB2 receptors, or both.

As used herein, “effective amount” refers to the administration of an amount of a given compound that achieves the desired effect. For example, regarding the combination of CBD and Δ⁸-THC, an “effective amount” is an amount sufficient for or that is capable of reducing nausea or vomiting and/or enhancing appetite in a patient or individual in need of such treatment. The patient may be a human patient.

As used herein, “purified” does not require absolute purity but is instead intended as a relative definition. For example, purification of starting material or natural material to at least one order of magnitude, preferably two or three orders of magnitude is expressly contemplated as falling within the definition of “purified.”

As used herein, the term “isolated” requires that the material be removed from its original environment.

As used herein, the terms “subject” and “patient” are used interchangeably irrespective of whether the subject has or is currently undergoing any form of treatment. As used herein, the terms “subject” and “subjects” may refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus monkey, chimpanzee, etc) and a human). The subject may be a human or a non-human. In this context, a “normal,” “control,” or “reference” subject, patient or population is/are one(s) that exhibit(s) no detectable disease or disorder, respectively.

“Sample,” “test sample,” and “patient sample” may be used interchangeably herein. The sample can be a sample of, serum, urine plasma, amniotic fluid, cerebrospinal fluid, cells (e.g., antibody-producing cells) or tissue. Such a sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. The terms “sample” and “biological sample” as used herein generally refer to a biological material being tested for and/or suspected of containing an analyte of interest such as antibodies. The sample may be any tissue sample from the subject. The sample may comprise protein from the subject.

The term “treating” or “treatment” refers to administration of a compound or agent to a subject who has a disorder or is at risk of developing the disorder with the purpose to cure, alleviate, relieve, remedy, delay the onset of, prevent, or ameliorate the disorder, the symptom of the disorder, the disease state secondary to the disorder, or the predisposition toward the disorder.

The terms “prevent,” “preventing,” “prevention,” “prophylactic treatment” and the like refer to reducing the probability of developing a disorder or condition in a subject (e.g., plant), who does not have, but is at risk of or susceptible to developing a disorder or condition.

The terms “decrease,” “reduced,” “reduction,” “decrease,” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition which is “substantially free” from Y may be completely free from Y. Where necessary, the word “substantially” may be omitted from the definition of the invention.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%1, 1%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise. In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.
It is understood that the examples and embodiments described herein are for illustrative purposes only and that various modifications or changes in light thereof will be suggested to persons skilled in the art and are to be included within the spirit and purview of this application and scope of the appended claims.

### E. EXAMPLES

### Example 1

To reduce cannabinoid (e.g., THC) loss and increase shelf life stability of cannabinoid compositions, various formulation methods and conditions have been investigated. For example, different formulations were tested to mitigate loss of the active pharmaceutical ingredient (API) such as THC, allowing for a shelf-stable infused beverage product over long time periods. Tocopherols (multiple forms of vitamin E) were emulsified along with cannabis oil for its natural antioxidant properties but was not found to suitably mitigate oxidative damage by itself. In follow-up trials, ascorbic acid (vitamin C, about 0.5-1 mM) was dissolved in the aqueous base to act as an aqueous-phase antioxidant and was found to provide ample protection to the cannabinoid API such as THC. Heat-accelerated experiments (30 days @ ˜37° C., equivalent to ˜10 months at room temperature) showed sufficient protection to THC and also strongly protected against unfavorable color and flavor alterations in the beverage product. Room temperature trials are ongoing, but extremely positive results have already been observed, as, after roughly five months, there were negligible amounts of THC loss with the addition of ascorbic acid compared to tocopherol-only preparations.

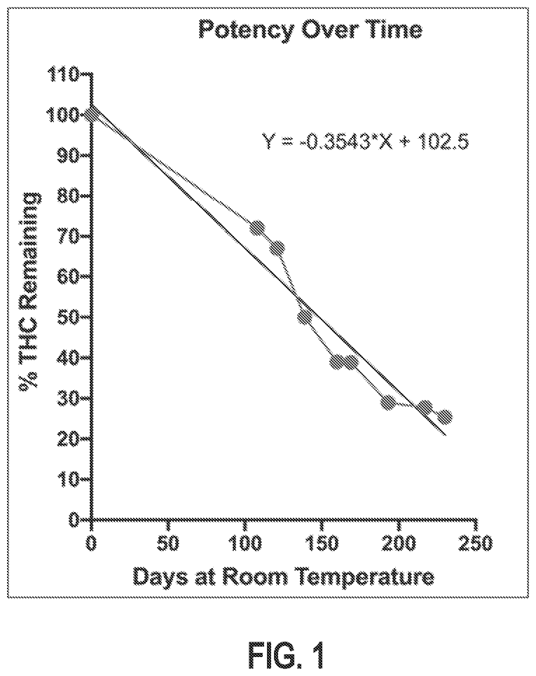

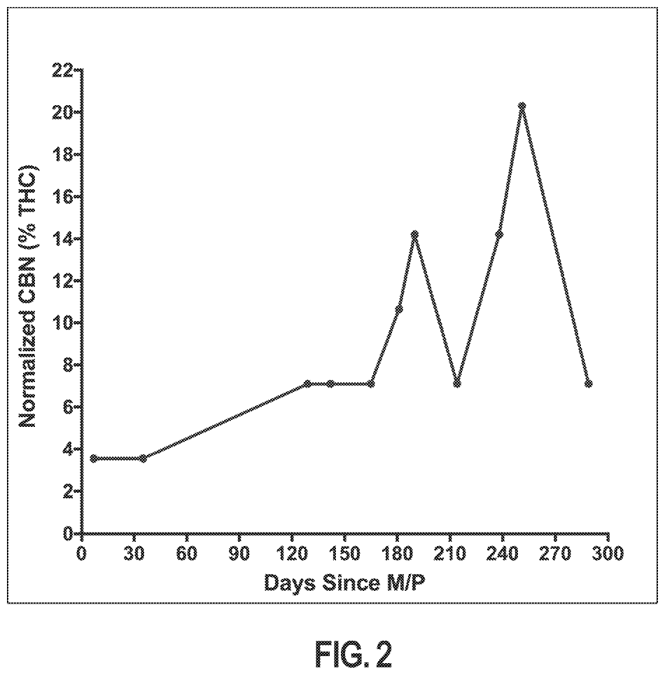

FIG. 1 is a graph showing quantified THC content in infused hop water beverages stored at room temperature. No antioxidants added. The rate of loss was found to be linear and roughly −0.35% THC per day. FIG. 2 is a graph showing quantified CBN (the canonical oxidation product of THC) in the room temperature infused hop water samples. CBN levels did rise over time, but not enough to fully account for the amount of THC that was lost. Maximal CBN levels observed were about 20% of the original THC content.

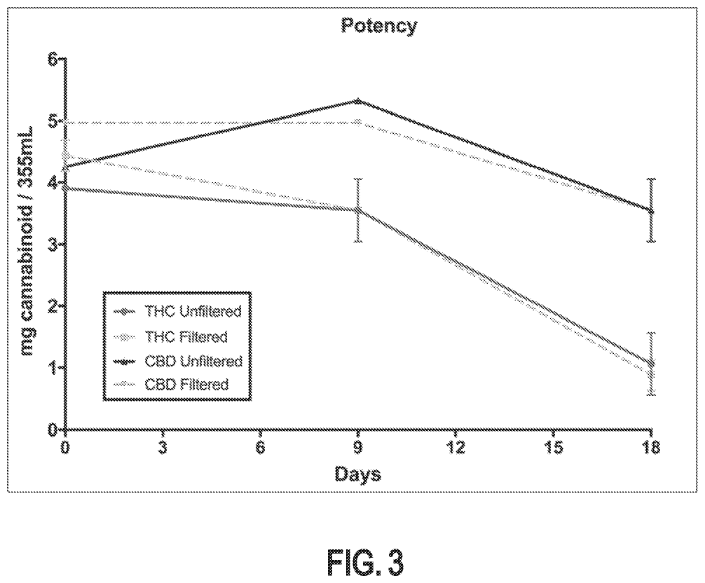

FIG. 3 is a graph showing an experiment ruling out any biological cause of cannabinoid loss in beverages. Infused hop water was dosed 5 mg THC+5 mg CBD. Compared conditions±pushing through a 0.22 μm syringe filter. No difference was observed between sterile-filtered and non-sterile conditions.

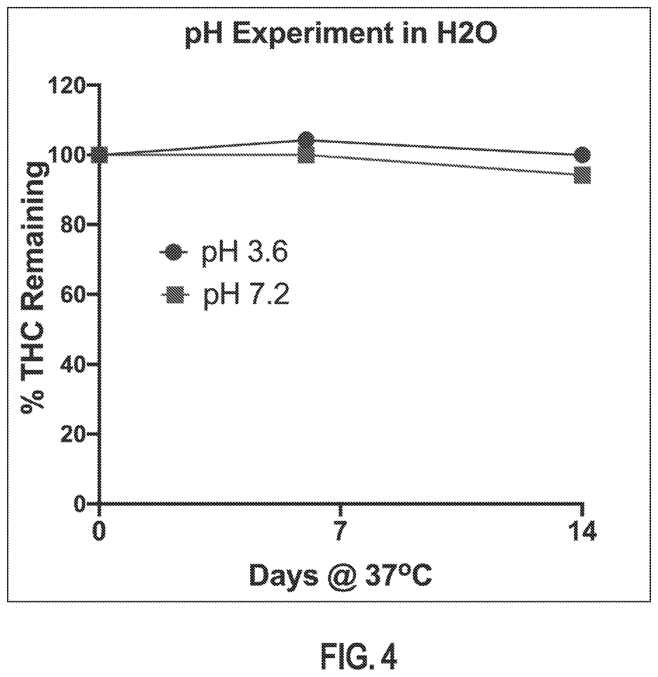

FIG. 4 is a graph showing an experiment ruling out pH as a driving factor behind THC loss. Dosed RO water to 20 mg THC/355 mL and used citric acid to pH down to 3.6 (which is the pH of the Infused hop water products) vs. neutral pH (had to bring H₂O pH up a bit using sodium carbonate). No difference was observed.

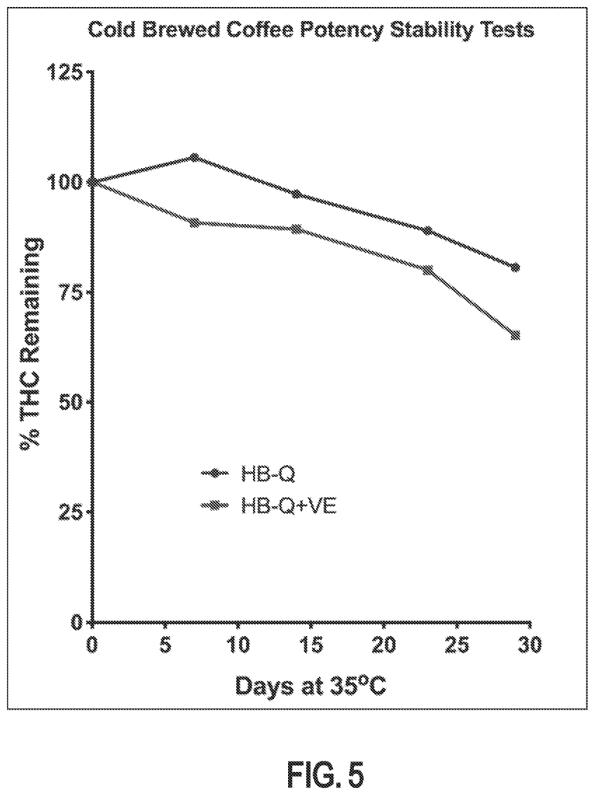

FIG. 5 is a graph showing quantified THC content when alpha-tocopherol (Vitamin E, VE) was incorporated into our emulsion alongside THC. Alpha-tocopherol (Vitamin E, VE) was incorporated into the emulsion alongside THC. Base used here is a cold-brewed coffee, which we know to exhibit far lower rates of THC loss compared to hop water (aka infused hop water) that was used for most other experiments. In this example, no appreciable difference between emulsion formulated with or without Vitamin E was observed.

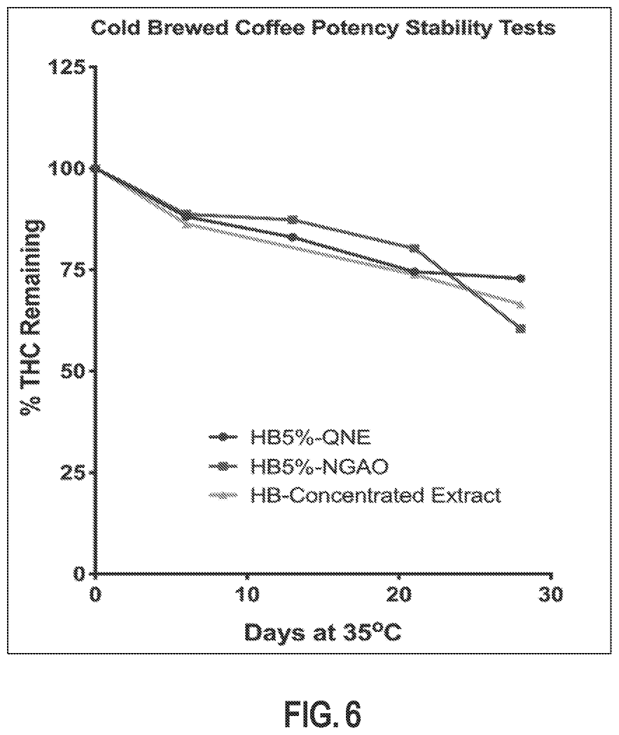

FIG. 6 is a graph showing cannabinoid stability testing on a cold-brewed coffee product (20× concentrated base and final product) using two different emulsions containing tocopherols (QNE=our in-house Q-naturale+VitE emulsion; NGAO=Nanogen's (a 3^{rd} party emulsion provider) antioxidant formulation). It was concluded that the in-house vitamin E-supplemented emulsion performs just as well as the antioxidant formulation that this emulsion-specializing company uses.

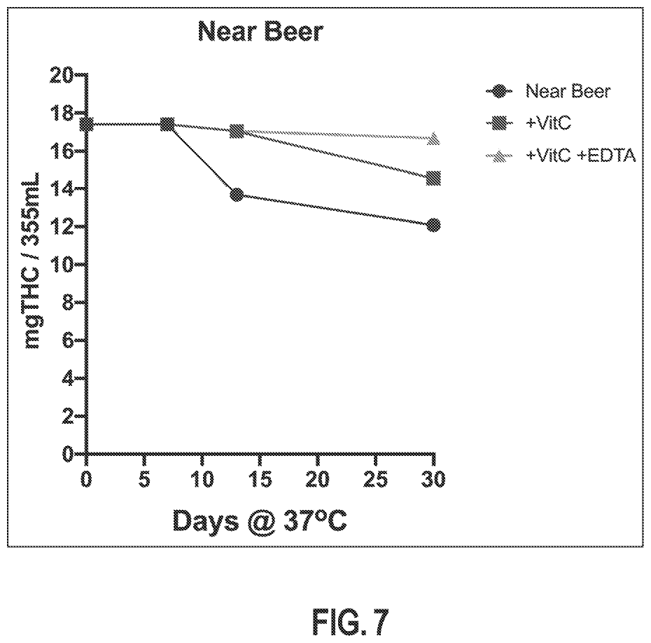

FIG. 7 is a graph showing quantified THC content when natural antioxidant ascorbic acid (Vitamin C, 176 ppm=1 mM) and/or metal chelator EDTA (25 ppm) was incorporated in the aqueous phase. The experiment was performed in a hop terpene-infused. Addition of vitamin C slowed rates of cannabinoid loss. No loss at all was detected when both vitamin C and EDTA were used.

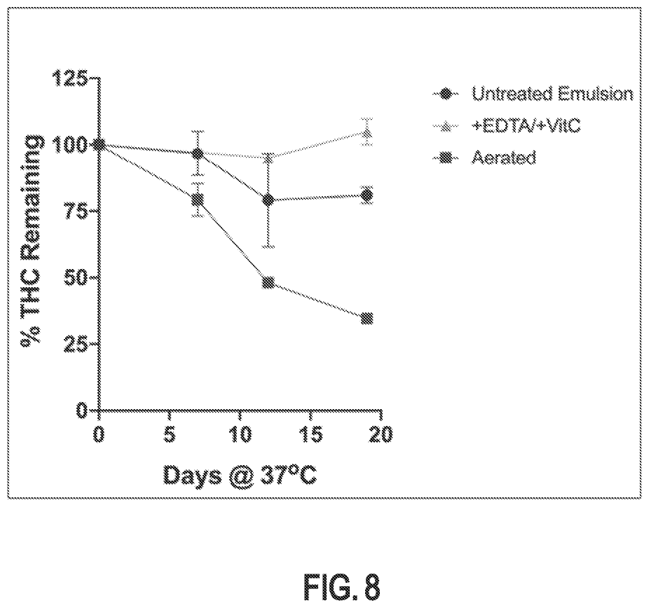

FIG. 8 is a graph showing quantified THC content when natural antioxidant ascorbic acid (Vitamin C, 176 ppm=1 mM) and/or metal chelator EDTA (25 ppm was incorporated in the aqueous phase. Aerated samples were shaken up over a few hours with air exposure to purposely increase dissolved oxygen (DO) levels. High DO samples showed the fastest rate of THC loss and partially helped to support that the mechanism of loss was via oxidation. Once again, no loss was observed in these heat-accelerated experiments when both vitamin C and EDTA were utilized.

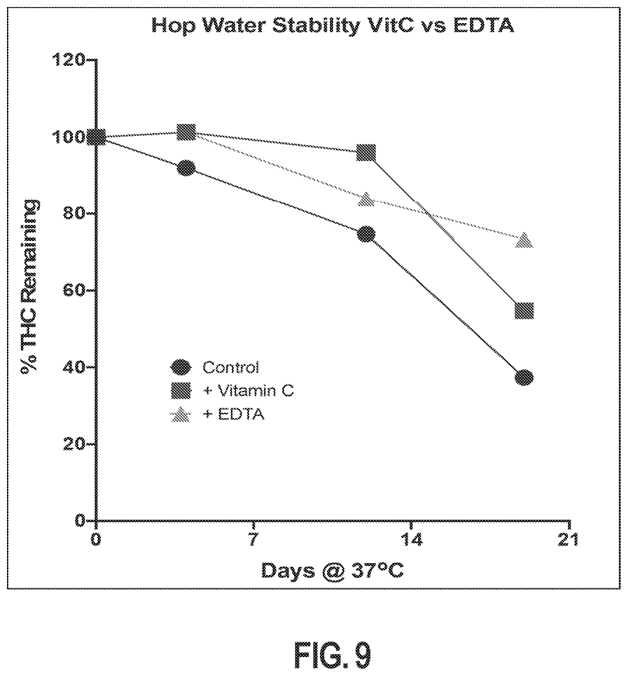

FIG. 9 is a graph showing quantified THC content in infused hop water products when vitamin C or EDTA was incorporated in the aqueous phase. The first experiment examined EDTA and vitamin C individually in the actual hop water base that is used for infused hop water products. Either vitamin C or EDTA was able to mitigate THC loss compared to control, but did not fully prevent it like was observed when using the combination of both.

Stability of infused hop water after 3 weeks at 37° C. incubation was tested. It was found addition of vitamin C could largely prevent formation of off-colors and flavors in the infused hop water products. Almost no alterations was found in color or flavor when a combination of EDTA (25 ppm) and vitamin C (176 ppm) was added.

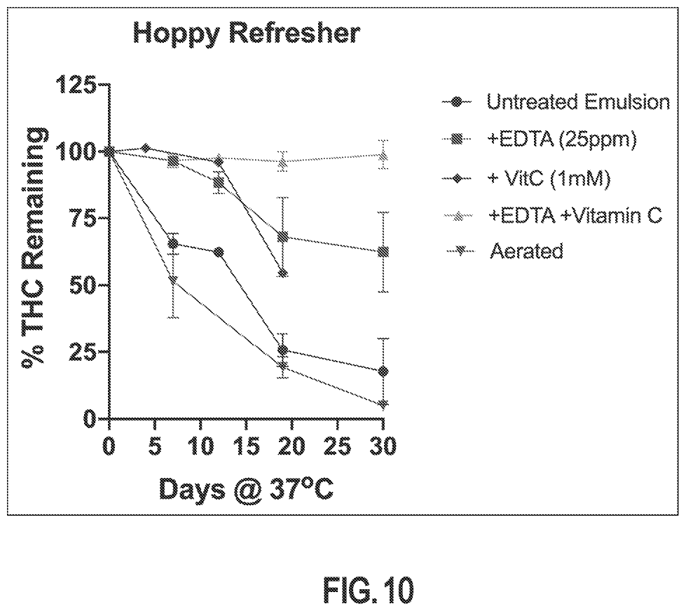

FIG. 10 is a graph showing combined data from heat-accelerated testing on three different emulsion formulations. A macroemulsion was compared to our in-house nanoemulsion and another nanoemulsion made by 3^{rd} party company Nanogen. Results were all very similar, so the datasets were combined together into a single figure. This dataset confirms the previous observations that vitamin C+EDTA confers outstanding protection against emulsified THC loss. Intentional increase in DO levels correlated to faster rates of loss (note: for comparison, the black ‘vitamin C-only’ dataset was overlaid on this figure from another experiment using our in-house emulsion that only went out 3 weeks).

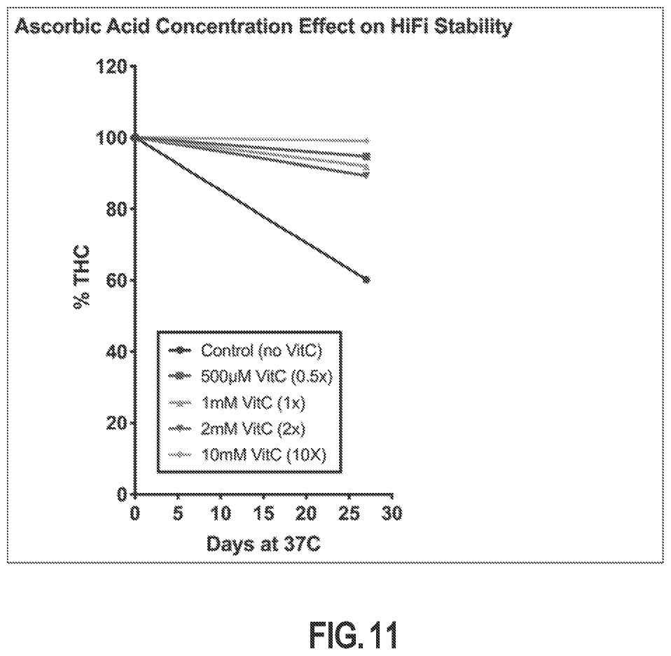

FIG. 11 is a graph showing quantified THC content in infused hop water products with different concentrations (0, 0.5 mM, 1 mM, 2 mM, and 10 mM) of vitamin C. Ascorbic acid is inherently tart, and the flavor was somewhat apparent in the final infused hop water product in blinded sensory panels. This experiment was conducted to determine if less vitamin C is sufficient to confer protection or if using more might lend even higher levels of protection. Interestingly, little difference was observed in antioxidant efficacy between 0.5-10 mM concentrations over 4 weeks of heat-accelerated testing (equivalent to ˜9 months at RT). It was concluded that the 500 μM concentration of vitamin is sufficient for infused hop water products.

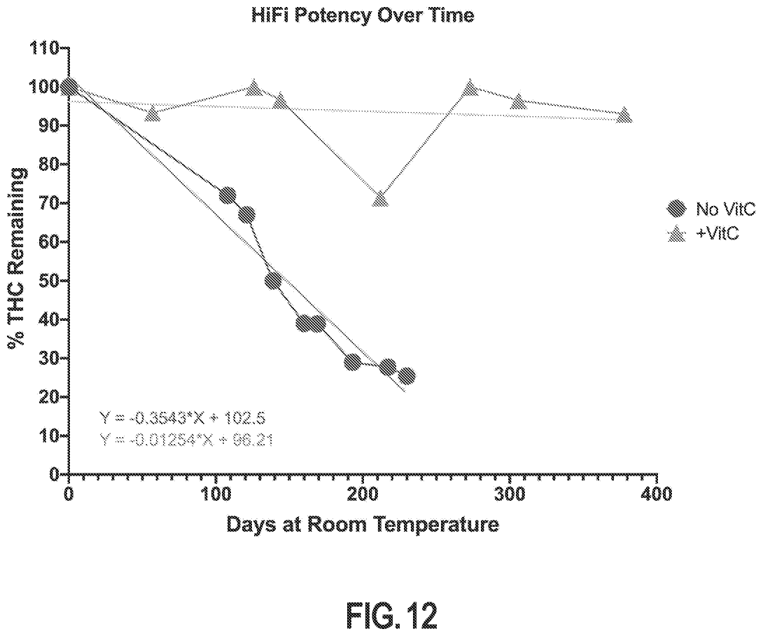

FIG. 12 is a graph showing quantified THC content in infused hop water products with 0.5 mM vitamin C. EDTA at the tested concentration would be an FDA-approved additive.

This disclosure also studied the effects of vitamin E when used in combination with vitamin C. Vitamin E (mixed tocopherol species, 20000 ppm or ˜2% of lipid phase) was added to the emulsion in combination with vitamin C for the condition portrayed in orange above, but neither antioxidant was used for the condition shown in green. This was conducted in hop water from real production runs of infused hop water (infused to 10 mg cannabinoid per 355 mL bottle) at room temperature and shows >100× improvement in the rate of THC loss with vitamin E in the lipid phase and the addition of 0.5 mM vitamin C to the aqueous medium.

What is claimed is:

1. A composition comprising one or more solubilized cannabinoids and at least one antioxidant, a metal chelator or a combination thereof.

2. The composition of claim 1, wherein the composition is formulated as an emulsion comprising a lipid phase and an aqueous phase, the lipid phase comprising at least a fraction of the one or more cannabinoids and the aqueous phase comprising a first antioxidant.

3. The composition of claim 2, wherein the one or more cannabinoids comprise THC.

4. The composition of claim 3, wherein the THC is in an amount of between about 1 ppm and about 70,000 ppm.

5. The composition of claim 2, wherein the first antioxidant comprises ascorbic acid.

6. The composition of claim 5, wherein the ascorbic acid is in an amount of between about 0.5 mM and about 1 mM of the aqueous phase.

7. The composition of claim 2, wherein the aqueous phase further comprises EDTA.

8. The composition of claim 7, wherein the EDTA is in an amount of between about 1 ppm and about 150 ppm.

9. The composition of claim 2, wherein the lipid phase comprises a second antioxidant.

10. The composition of claim 9, wherein the second antioxidant comprises tocopherol.

11. The composition of claim 10, wherein the tocopherol is in an amount of between about 2% and about 5% by weight of the lipid phase.

12. The composition of claim 10, wherein the composition comprises:
between about 0.5 mM and about 1 mM of the ascorbic acid;
between about 1 ppm and about 150 ppm of the EDTA; and
between about 2% and about 5% by weight of the tocopherol.

13. The composition of claim 2, wherein the emulsion comprises emulsion droplets having a mean diameter of between about 50 nm and about 800 nm.

14. The composition of claim 1, wherein the one or more cannabinoids are solubilized through cyclodextrin-mediated encapsulation, THC-glycosylation, or a protein-mediated carrier.

15. The composition of claim 1, further comprising an additive, a pharmaceutically acceptable carrier, an adjuvant or a second agent.

16. The composition of claim 15, wherein the second agent is selected from the group consisting of: cannabinoids, terpenes, anti-insomnia, anti-tussive, opioid analgesic, decongestant, non-opioid analgesic/anti-inflammatory drug, anti-migraine drug, anti-emetic, anti-histamine, proton pump inhibitor, H2 antagonist/H2 blocker, tranquilizer, anticonvulsant, hypnotic, muscle relaxant, anti-psychotic, anti-diarrheal, attention deficit and hyperactivity disorder (ADHD) drug, anti-Parkinson disease drug, benzodiazepine, benzodiazepine antagonist, barbiturate, barbiturate antagonist, stimulant, stimulant antagonist, antidepressant, nutraceutical, nicotine, BCS Class II active ingredient, BCS Class IV active ingredient, anti-multiple sclerosis (MS) drug, ethyl pyruvate, melatonin, caffeine, resveratrol, and a combination thereof.

17. The composition of claim 1, wherein the composition is an oral dosage composition, a pulmonary or nasal dosage composition, or a topical dosage composition.

18. An edible product comprising the composition of claim 1.

19. A kit comprising the composition of claim 1.

20. A method of treating a subject, comprising administering to a subject afflicted with or suffering from nausea, muscular spasms, multiple sclerosis, uterine cramps, bowel cramps, a movement disorder, pain, migraine headache, glaucoma, asthma, inflammation, insomnia, high blood pressure, cancer, anxiety, convulsions, depression or psychosis, an effective amount of the composition of claim 1.
